(19) United States
(12) Patent Application Publication
(10) Pub. No.: US 2010/0098739 A1
(43) Pub. Date: Apr. 22, 2010

(54) COMPOSITIONS AND METHODS FOR MODULAR SOFT TISSUE REPAIR

(75) Inventors: Adam J. KATZ, Charlottesville, VA (US); Hulan SHANG, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/580,419

(22) Filed: Oct. 16, 2009

Related U.S. Application Data
(60) Provisional application No. 61/106,758, filed on Oct. 20, 2008.

(51) Int. Cl. A61F 2/00; C12N 11/00; A61K 35/12

(52) U.S. Cl. 424/423; 435/174; 424/93.7

(57) ABSTRACT

Adipose tissue-derived stromal cells induced to form multicellular aggregates can be formed reliably and consistently, they can be maintained for prolonged periods in adherent or suspension culture, and they are able to survive, grow, and/or differentiate in serum-free media conditions. The present invention provides compositions and methods for the use of such aggregates in tissue repair. This culture platform provides a controlled and defined system in which to study and standardize adult stem cell biology, and begets an instinctive “modular” approach to the predictable replacement and regeneration of adipose tissue.

### CROSS-REFERENCE TO RELATED APPLICATIONS

This application claims the benefit of U.S. Provisional Application No. 61/106,758, filed Oct. 20, 2008, the disclosure of which is incorporated herein by reference in its entirety.

### STATEMENT REGARDING FEDERALLY SPONSORED RESEARCH

This invention was made in part with United States Government support under National Institutes of Health Grant No. R21 HL72141. The United States Government has certain rights in the invention.

### BACKGROUND

The need to regenerate and/or replace adipose tissue for soft tissue reconstruction is often minimized or completely overlooked. The reconstructive surgeon, however, realizes the importance, as well as the unique challenge of this clinical objective. Soft tissue reconstruction is an integral aspect of surgical approaches to limb amputation, burn injury, and facial trauma. In these cases, contour deformity and/or poor skin and scar quality could be improved by reliable replacement of subcutaneous adipose tissue. Similarly, oncologic resections often require soft tissue replacement/regeneration to help reestablish a patient's self-image. Breast reconstruction after mastectomy is a prime example of this clinical scenario (Missana et al. 2007; Shenaq and Yuksel 2002). Likewise, craniofacial procedures for congenital malformations (such as hemifacial microsomia, or Romberg's progressive facial hemiatrophy), traumatic defects (avulsions, complex wounds), and neck dissections present similar reconstructive challenges due to a lack of soft tissue “fill” and/or symmetry.

The current standard of care for adipose/soft tissue replacement and regeneration has relied on three approaches: (1) surgical flaps that move adipose tissue from one site to another while maintaining an intact blood supply. These are associated with medical risks, high costs, scarring, and functional loss; (2) artificial fillers, such as Teflon paste, silicone implants, and bovine collagen, that lack any metabolic activity and risk migration, extrusion or allergic reactions, and; (3) Autologous Fat Transfers (AFT) that involve the transplantation of autologous adipose tissue fragments without an intact blood supply. Although AFT has undergone few changes since it was first introduced over a century ago, physicians performed nearly 500,000 procedures during the past year. However, the procedure remains more of an art than a science, and a significant need exists to improve its predictability and reproducibility. More often than not, AFT grafts lose their volume over time, and it is difficult to predict in advance the extent to which this will occur in any given patient. This has been attributed to traumatic rupture, avascular necrosis, and apoptosis of the adipocytes, inflammation secondary to cell death, fibrosis and contraction of the graft, and/or delipidation of the adipocytes with subsequent volume loss (Eremia and Newman 2000; Kanchwala and Bucky 2003; Kaufman et al. 2007).

Tissue engineering approaches to adipose replacement have become common in recent years, with a goal to provide predictable and reproducible platforms based on scientific principles. At present, all of these strategies are limited in the amount (size) of tissue that can be re(generated) in one sitting due to limitations in vascular supply. Nevertheless, the development of effective and utilitarian adipose replacement techniques for even small volumes would be a significant advancement in the field; and, as parallel integrative technologies advance, would likely provide a platform for the future treatment of larger and larger tissue volumes. The current general paradigm for these engineered approaches involve the use of progenitor and/or stem cells with adipogenic potential in conjunction with matrix carriers or bioengineered scaffolds (Choi et al. 2005; Halbleib et al. 2003; Hemmrich and von Heimburg 2006; Kimura et al. 2003; Prichard et al. 2007). Whereas a wide variety of natural and synthetic scaffold materials and delivery agents have been explored, adipose derived stem cells (often interchangeably referred to as stromal, progenitor or preadipocyte cells) are generally regarded as the logical and ideal cell source for these objectives. The fact that none of the numerous scaffold-based technologies for soft tissue regeneration has yet made it to the clinic reflects both the extent of the challenge at hand and the continued need for more effective strategies.

There is a long felt need in the art for compositions and methods useful for regenerating adipose tissue for use in soft tissue reconstruction. The present invention satisfies this need.

### SUMMARY OF THE INVENTION

In order to regenerate adipose tissue effectively, it makes perfect sense to use cellular components derived from the target tissue and embed them into a self-generated matrix that resembles their endogenous microenvironment. It is reasonable to believe that a graft/implant engineered in this manner would display superior in vivo engraftment and tissue integration in the near term, and subsequent volume retention, biological function, and appropriate remodeling over the long term.

In one embodiment, the present invention provides compositions and methods useful for the three-dimensional organization and maintenance of human ASCs in a self-generated extracellular matrix. The ASCs maintain the ability to proliferate and to differentiate. In one aspect, the present invention provides methods for administering these three-dimensional constructs to subjects in need thereof. In one aspect, the invention provides a novel platform for modular soft tissue regeneration. In one aspect, no exogenous materials or scaffolds are required.

In accordance with one embodiment a method of making a 3-dimensional modular adipogenic construct is provided. The modular adipogenic construct includes cellular components with adipogenic potential (including adipose stem cells) and has a self-generated extracellular matrix. Furthermore, the modular adipogenic construct is serum-free, free of exogenous materials, xenogenic-free, and free of other synthetic components. This basic modular construct is prepared by harvesting adipose tissue from a mammalian subject, isolating adipose tissue-derived stromal cells from the harvested adipose tissue, and culturing the isolated cells in 3-dimensional multicellular aggregates in a controlled, reproducible fashion. Culturing of isolated cells means the in vitro culturing of the isolated adipose tissue-derived stromal cells using appropriate means and methods. In one embodiment the adipose tissue is isolated from a human and in one embodiment the adipose tissue is harvested from the same individual that will receive a subsequently formed 3-dimensional multicellular aggregate implant derived from the harvested adipose tissue.

The preparation of the multicellular aggregate can be prepared as described herein and in previous patent applications: U.S. Provisional Application Nos. 61/221,577, filed Jun. 30, 2009; 61/118,055, filed Nov. 26, 2008; 61/107,398, filed Oct. 22, 2008; U.S. patent application Ser. No. 12/444,412, filed Apr. 6, 2009 and International application no. PCT/US2009/033220, filed Feb. 5, 2009 (published on Aug. 13, 2009 as WO 2009/100219), the disclosures of which are incorporated herein by reference in their entirety. In accordance with one embodiment, harvesting adipose tissue means the surgical removal of adipose tissue from other tissues naturally associated tissues resulting in a substantial enrichment of adipose tissue. In one embodiment harvesting adipose tissue is conducted either by excision, or more commonly, by liposuction. Isolation of adipose tissue-derived stromal cells from adipose tissues includes enriching for stromal cells relative to the harvested adipose tissue. Isolation of stromal cells can be conducted using mechanical and/or chemical processes by which the adipose tissue-derived stromal cells are separated (isolated) from the harvested adipose tissue. Typically the isolated stromal cells are cultured prior to formation of the 3-dimensional modular adipogenic constructs disclosed herein and such culturing steps include the in vitro culturing of the isolated adipose tissue-derived stromal cells using appropriate means and methods.

A 3-dimensional culture is a culture of cells that has an additional dimension, a ‘depth dimension’, over and above the traditional 2-dimensional adherent monolayer cell culture. Advantageously, the 3-dimensional modular adipogenic constructs can be combined with isolated cells or cellular components that are selected based on the intended implantation site of the construct into a mammalian host. Such components include purified collagen or other extracellular components, growth factors, antibiotics, or cytokines.

In accordance with one embodiment a method is provided for inducing or enhancing the repair of damaged or diseased tissues through the use of the disclosed 3-dimensional modular adipogenic constructs. Such methods include stimulating growth, proliferation or differentiation of tissues to enhance tissue repair, tissue regeneration, tissue replacement, or tissue augmentation. In one embodiment the method comprises the steps of generating 3-dimensional modular adipogenic constructs, and implanting said modular adipogenic constructs in the tissues of a patient at a site in need of tissue regeneration or repair. In further embodiments of the invention the modular adipogenic construct is combined with exogenous bioactive agents or cells, including for example, growth factors, antibiotics, or cytokines. In addition, in some embodiments the adipogenic construct is combined with a gel, scaffold, or exogenous factor.

Various aspects and embodiments of the invention are described in further detail below.

### BRIEF DESCRIPTION OF THE DRAWINGS

FIG. 1A-C: Formation and 3-Dimensional (Suspension) Culture of Human ASC Aggregates. (FIG. 1A) ASC multicellular aggregates (MAs) are reproducibly formed using a hanging droplet technique. After 24-72 hours in a hanging droplet, they can be reliably transferred to non-adherent culture-ware for 3-D culture, (or to regular culture-ware for adherent culture). FIG. 1B shows multiple, uniform-sized ASC MAs prepared using cells pre-labeled with DiI fluorescent dye, maintained in 3-D culture. In FIG. 1C, cell nuclei have been stained blue in a MA in 3-D culture, providing additional perspective on the cellular topography of these aggregates.

FIGS. 2A&B: ASC MAs are Composed of Cells and Cell-generated Extracellular Matrix. ASCs were prepared as MAs and maintained in 3-D culture. Histology of stained sections demonstrates the presence of abundant cellularity (ASCs) embedded within a self-generated extracellular matrix, composed of abundant collagen. FIG. 2A shows H & E staining, FIG. 2B shows Trichrome staining (100×).

FIG. 3A-D: ASC Proliferation Within MAs In Serum Free, 3-D Culture. (FIG. 3A) Light micrograph of an ASC MA grown in suspension culture in serum-free medium (AR9), with early evidence of polarization and directional growth. (FIG. 3B) H&E histology of the same MA demonstrates defined nuclei throughout a background of abundant ASC-derived extracellular matrix. (FIG. 3C) Fluorescent micrograph of the same section in (FIG. 3B) demonstrates the more intense, cell and matrix-dense ‘core’ relative to the growing ‘apical’ edge (toward the right). (FIG. 3D) Reveals a light micrograph of a section from the same MA that has been immunohistochemically stained for BrdU (brown nuclei), demonstrating viable, replicating ASCs within the center and edge of the niche. The MA was grown in serum-free medium for 8 days, and pulsed with BrdU for 24 hours prior to fixation and staining. The observed staining pattern correlates perfectly with the proliferative activity observed at the non-fluorescent pole (100×).

FIG. 4: No figure provided for this figure number.

FIG. 5: Adipogenic Differentiation of ASC MAs in 3-D (Suspension) Culture. Human ASC MAs were cultured for 2 weeks in serum-free medium (AR9) and then exposed to adipogenic medium for 3 weeks. Control MAs remained in AR9 medium for a total of 5 weeks. Oil Red O staining of control (top left) and adipo-induced (bottom left) specimens demonstrates the notable presence of lipid in the latter. (100×) Quantitative RT-PCR analysis reveals dramatic upregulation of adipose-related genes at the 3-week timepoint in MAs exposed to induction medium, supporting the histological findings. Values represent the average fold change of gene expression relative to control MAs/conditions (N=3 different donors).

FIG. 6: Differentiation of Human ASC MAs after Placement in Adherent Culture. ASC MAs were cultured for 4 weeks in serum-free suspension culture, then placed into adherent culture (tissue culture plastic) and adipogenic induction medium for 2 weeks. Cultures were then fixed and stained with Oil Red O for lipid. Note the presence of free lipid droplets in the medium (arrow), indicative of adipogenic differentiation. (40×, left; 100×, right).

FIG. 7: Differentiation of 2-D Monolayer ASCs Derived from Adherent, Serially Passaged MAs. Human ASC MAs were maintained for 2 weeks in serum-free 3-D culture, and then placed into adherent culture (tissue culture plastic) in D-10 medium. The adherent MAs were passaged every 3-4 days to new culture wells 10 separate times. Passage 10 MA-derived cells were then expanded to near-confluence in D-10 medium over 6 weeks. The expanded P10 cells were then cultured in adipogenic induction medium for 2 weeks. Cells were then fixed and stained with Oil Red O for lipid. Image on left shows entire culture well, while image on right shows representative microscopic view. (3.5×, left; 60×, right).

FIG. 8: No figure provided for this figure number.

FIGS. 9A-C: ASCs Formulated as 3-D Aggregates Can Be Successfully Delivered In Vivo. FIGS. 9A and 9B show human ASCs MAS 2 weeks after injection into the left ventricular wall of a mouse. The cells were labeled with DiI and can be visualized by fluorescent microscopy (FIG. 9B). Image FIG. 9C shows ASC MAs after placement into a full thickness cutaneous wound. The aggregates are grossly visible (black arrows).

FIGS. 10-19: Represent various confocal images of tissues harvested after administration of the 3-dimensional modular adipogenic constructs of the present invention.

### DETAILED DESCRIPTION

### Abbreviations and Acronyms

ASC—adipose tissue-derived stem cell

ASCB—adipose stem/stromal cell blastema

ASC-MB—ASC-mesenchymal blastema or mesenchoid body

CB—chimeric blastema

DMEM—Dulbecco's modified Eagle's medium

ECM—extracellular matrix

ES—embryonic stem cell

FACS—fluorescent activated cell sorting

FBS—fetal bovine serum

FGF—fibroblast growth factor

gf—growth factor

HSC—hematopoietic stem cell

HS—human serum (also referred to as HmS herein)

HSA—human serum albumin

IL-1β—interleukin-1 beta

MB—mesenchoid body

PDGF—platelet-derived growth factor

PLA—processed lipoaspirate cells

SCGF-β—stem cell growth factor-β

SFM—serum-free medium (also referred to as sf herein)

SNiM—Self-organizing Niche Milieu, which is another term for ASC aggregates

SOM-B—Self-Organizing Mesenchymal Blastema (also referred to as “self-organizing mesenchoid bodies” and as SNiM herein)

TNFα—tumor necrosis factor alpha

ULA—ultra low attachment tissue culture plate

VEGF—Vascular endothelial growth factor

### Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease or disorder, including, but not limited to, pain and inflammation.

“Adipose-derived stem cells”, also referred to as “adipose-derived stromal cells” herein, refer to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, more preferably, the adipose tissue is human. A convenient source of adipose tissue is from liposuction surgery, however, the source of adipose tissue or the method of isolation of adipose tissue is not critical to the invention.

The term “adult” as used herein, is meant to refer to any non-embryonic or non-juvenile subject. For example the term “adult adipose tissue stem cell,” refers to an adipose stem cell, other than that obtained from an embryo or juvenile subject.

A disease or disorder is “alleviated” if the severity of a symptom of the disease, condition, or disorder, or the frequency with which such a symptom is experienced by a subject, or both, are reduced.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

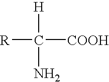

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients may be added. The term does not exclude media which have been prepared or are intended for specific uses, but which upon modification can be used for other cell types, etc.

The term “blastema”, as used herein, encompasses inter alia, the primordial cellular mass from which an organ, tissue or part is formed as well as a cluster of cells competent to initiate and/or facilitate the regeneration of a damaged or ablated structure.

The term “biocompatible,” as used herein, refers to a material that does not elicit a substantial detrimental response in the host.

The term “biodegradable,” as used herein, means capable of being biologically decomposed. A biodegradable material differs from a non-biodegradable material in that a biodegradable material can be biologically decomposed into units which may be either removed from the biological system and/or chemically incorporated into the biological system.

The term “bioresorbable,” as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable,” as the term is used herein, means that at least 80% of the total mass of material implanted is resorbed within one year.

The terms “cell” and “cell line,” as used herein, may be used interchangeably. All of these terms also include their progeny, which are any and all subsequent generations. It is understood that all progeny may not be identical due to deliberate or inadvertent mutations.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “compound,” as used herein, refers to any type of substance or agent that is commonly considered a drug, or a candidate for use as a drug, combinations, and mixtures of the above, as well as polypeptides and antibodies of the invention.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The term “delivery vehicle” refers to any kind of device or material which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. Feeder cells can be non-lethally irradiated or treated to prevent their proliferation prior to being co-cultured to ensure to that they do not proliferate and mingle with the cells which they are feeding. The terms, “feeder cells”, “feeders,” and “feeder layers” are used interchangeably herein.

As used herein, a “functional” molecule is a molecule in a form in which it exhibits a property or activity by which it is characterized.

A “fragment” or “segment” is a portion of an amino acid sequence, comprising at least one amino acid, or a portion of a nucleic acid sequence comprising at least one nucleotide. The terms “fragment” and “segment” are used interchangeably herein.

“Graft” refers to any free (unattached) cell, tissue, or organ for transplantation.

“Allograft” or “allogeneic” refers to a transplanted cell, tissue, or organ derived from a different animal of the same species.

“Xenograft” or “xenogeneic” refers to a transplanted cell, tissue, or organ derived from an animal of a different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “ingredient” refers to any compound, whether of chemical or biological origin, that can be used in cell culture media to maintain or promote the proliferation, survival, or differentiation of cells. The terms “component,” “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit,” as used herein, means to suppress or block an activity or function such that it is lower relative to a control value. The inhibition can be via direct or indirect mechanisms. In one aspect, the activity is suppressed or blocked by at least 10% compared to a control value, more preferably by at least 25%, and even more preferably by at least 50%.

The term “inhibitor” as used herein, refers to any compound or agent, the application of which results in the inhibition of a process or function of interest, including, but not limited to, differentiation and activity Inhibition can be inferred if there is a reduction in the activity or function of interest.

The term “injury” refers to any physical damage to the body caused by violence, accident, trauma, or fracture, etc.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

Used interchangeably herein are the terms: 1) “isolate” and “select”; and 2) “detect” and “identify”.

The term “isolated,” when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays which distinguish cell types.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “low adherence, ultra low adherence, or non-adherence surface for cell attachment” refers to the ability of a surface to support attachment of cells. The term “non-adherence surface for cell attachment” means that the surface supports little if any cell attachment.

The term “modulate”, as used herein, refers to changing the level of an activity, function, or process. The term “modulate” encompasses both inhibiting and stimulating an activity, function, or process.

The terms “multicellular aggregate”, “multicellular sphere”, “blastema”, and “multicellular structure” are used interchangeably herein.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Plurality” means at least two.

The term “progeny” of a stem cell as used herein refers to a cell which is derived from a stem cell and may still have all of the differentiation abilities of the parental stem cell, i.e., multipotency, or one that may no longer be multipotent, but is now committed to being able to differentiate into only one cell type, i.e., a committed cell type. The term may also refer to a differentiated cell.

The term “propagate” means to reproduce or to generate.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

As used herein, the term “solid support” when used in reference to a substrate forming a linkage with a compound, relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “solid support suitable for maintaining cells in a tissue culture environment” is meant any surface such as a tissue culture dish or plate, or even a cover, where medium containing cells can be added, and that support can be placed into a suitable environment such as a tissue culture incubator for maintaining or growing the cells. This should of course be a solid support that is either sterile or capable of being sterilized. The support does not need to be one suitable for cell attachment.

The term “solid support is a low adherence, ultralow adherence, or non-adherence support for cell culture purposes” refers to a vehicle such as a bacteriological plate or a tissue culture dish or plate which has not been treated or prepared to enhance the ability of mammalian cells to adhere to the surface. It could include, for example, a dish where a layer of agar has been added to prevent cells from attaching. It is known to those of ordinary skill in the art that bacteriological plates are not treated to enhance attachment of mammalian cells because bacteriological plates are generally used with agar, where bacteria are suspended in the agar and grow in the agar.

The term “spawn”, as used herein, refers to the ability of the multicellular spheres of cells disclosed herein (SOMBs) to generate adherent cells (i.e., progeny) with the ability, inter alia, to grow to confluence.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In one aspect, the activity or differentiation is stimulated by at least 10% compared to a control value, more preferably by at least 25%, and even more preferably by at least 50%. The term “stimulator” as used herein, refers to any compound or agent, the application of which results in the stimulation of a process or function of interest, including, but not limited to, ASC cell production, differentiation, and activity, as well as that of ASC progeny.

A “subject” of analysis, diagnosis, or treatment is an animal. Such animals include mammals, preferably a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “substituent” as used in the phrase “other cells which are not substituents of the at least one self-organizing blastema” refers to substituent cells of the blastema. Therefore, a cell which is not a substituent of a self-organizing blastema can be a cell that is adjacent to the blastema and need not be a cell derived from a self-organizing blastema.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The use of the phrase “tissue culture dish or plate” refers to any type of vessel which can be used to plate cells for growth or differentiation.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

As used herein, the term “wound” relates to a physical tear, break, or rupture to a tissue or cell layer. A wound may occur by any physical insult, including a surgical procedure or as a result of a disease, disorder condition.

### Embodiments

Methods useful for the practice of the invention which are not described herein are also known in the art. Useful methods include those described in WO 2007/030652 (PCT/US2006/034915), WO 2007/019107 (PCT/US2006/029686), WO 2007/089798 (PCT/US2007/002572), and WO 2008/060374 (PCT US2007/021432), the methods of which are hereby incorporated by reference.

We have developed reproducible methods of culture that support the 3-dimensional (3-D) organization and maintenance of human ASCs in a self-generated extracellular matrix. We refer to these multicellular aggregates as “MAs”, and in the setting of adipose tissue engineering, view them as “Modular Adipogenic Constructs”. In essence, each aggregate is a ‘micro-organoid’, or ‘regenerative nidus’ that can initiate and participate in tissue repair and replacement.

Human ASC MAs self-organize and survive in a variety of culture conditions, including defined serum-free conditions. In addition, human ASC MAs demonstrate proficient adipogenic potential in vitro and can be implanted in vivo without the need for exogenous materials or scaffolds. We therefore believe that ASC MAs are a novel and flexible platform for modular soft tissue regeneration. Furthermore, we anticipate that ASCs formulated and delivered as MAs will exhibit efficient survival after in vivo implantation, as well as effective and reliable adipogenesis for long-term volume maintenance.

Adipose tissue (fat) embodies all of the requisite qualities of an ideal autologous cell source. With the prevalence of obesity reaching epidemic proportions in the US and globally, adipose tissue is abundant, expendable and replenishable (Kumanyika 2007;Wang and Beydoun 2007;Wilmore 2007). In addition, it is safe and easy to harvest through minimally invasive aspiration/suction techniques, a procedure actually perceived by many as appealing. Recent work shows that an average of 400,000 ASCs can be obtained from every milliliter of suction-harvested adipose tissue (Aust et al. 2004). Given the fact that up to two liters of adipose tissue can be safely removed from an individual with a single, minimally invasive outpatient procedure, nearly 800 million ASCs can be readily obtained with minimal morbidity. These unmatched qualities make ASCs practical and ideally suited for translational self-cell therapies.

Several independent research groups, including my own, have now published on the characterization, self-renewal and multilineage developmental plasticity of stem (stromal) cells isolated from human subcutaneous adipose tissue. Their developmental spectrum includes lineages characterized as osteogenic, chondrogenic, myogenic, angiogenic, neurogenic (Gimble et al. 2007; Parker and Katz 2006; Schaffler and Buehler 2007) and most pertinent to this proposal, adipogenic. In addition to their developmental plasticity, human ASCs are also known to produce a wide variety of soluble and insoluble factors that favorably impact the repair of damaged tissues through the modulation of angiogenesis, inflammation, apoptosis, cell homing, cell proliferation, and cell migration

(Gimble et al. 2007;Parker and Katz 2006). For example, Rehman et al. have shown that hASCs secrete several angiogenic and anti-apoptotic proteins that can be up-regulated under hypoxic and other culture conditions (Rehman et al. 2004). Angiogenesis is known to be tightly and critically linked to adipogenesis (Cao 2007;Crandall et al. 1997;Fukumura et al. 2003;Lijnen 2007), and our preliminary data show that ASCs secrete higher levels of many growth factors (such as VEGF and HGF) when formulated as MAs compared to similar cells grown in traditional monolayer culture. We propose that such ‘biological enhancement’ is largely related to the 3-D nature of MAs, which more accurately reflects in vivo conditions (i.e. niches) than does 2-D monolayer culture on plastic or other synthetic scaffolds.

The vast majority of research related to mesenchymal stem cell populations in general, and adipose stem cells (ASCs) in particular, has centered on their biology, behavior and therapeutic potential as adherent monolayer cell cultures. All adult tissues, including fat tissue, exist in a homeostatic equilibrium under baseline conditions, with cell loss being balanced by the generation of new cells. New cells are generated by the stem/progenitor cell reservoir of a tissue, which exists in vivo within the context of a 3-D supportive niche, or microenvironment. As reviewed in several recent papers, a stem cell niche is composed of a diverse collection of cells (in addition to, or including the stem cell constituents) that provide a milieu of soluble and matrix factors. These factors, in turn, define a microenvironment that directs and controls the homeostasis of the stem cell niche—including cell growth, cell differentiation, and cell renewal (Fuchs et al. 2004; Kindler 2005). In short, “it is the combination of the intrinsic characteristics of (stem) cells and their microenvironment that shapes their properties and defines their potential” (Fuchs et al. 2004). The niche/micromilieu concept is uncannily reflected in the process of in vivo adipose tissue development. Nishimura et al. demonstrated that in vivo adipogenesis initiates from “cell clusters” that are composed of multiple cell types including stromal cells and endothelial cells (Nishimura et al. 2007). ASC MAs embody ex vivo engineered analogs of these “clusters” that occur naturally in vivo, and we believe they will drive the generation of new adipose tissue when implanted in vivo.

Adult human extramedullary adipose tissue-derived stromal cells represent a stromal stem cell source that can be harvested routinely with minimal risk or discomfort to the patient. Pathologic evidence suggests that adipose-derived stromal cells are capable of differentiation along multiple lineage pathways. Adipose tissue is readily accessible and abundant in many individuals.

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there are up to 8.6×10⁴ stromal cells per gram of adipose tissue (Sen et al., 2001, Journal of Cellular Biochemistry 81:312-319). Ex vivo expansion over 2 to 4 weeks yields up to 500 million stromal cells from 0.5 kilograms of adipose tissue. These cells can be used immediately or cryopreserved for future autologous or allogeneic applications.

Adipose derived stromal cells also express a number of adhesion and surface proteins. These include, but are not limited to, cell surface markers such as CD9; CD29 (integrin beta 1); CD44 (hyaluronate receptor); CD49d,e (integrin alpha 4, 5); CD54 (ICAM1); CD55 (decay accelerating factor); CD105 (endoglin); CD106 (VCAM-1); CD166 (ALCAM) and HLA-ABC (Class I histocompatibility antigen); and cytokines such as interleukins 6, 7, 8, 11; macrophage-colony stimulating factor; GM-colony stimulating factor; granulocyte-colony stimulating factor; leukemia inhibitory factor; stem cell factor and bone morphogenetic protein. Many of these proteins have the potential to serve a hematopoietic supportive function and all of them are shared in common by bone marrow stromal cells.

The adipose tissue-derived stromal cells useful in the methods of invention can be isolated by a variety of methods known to those skilled in the art such as described in WO 00/53795. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject to provide for an autologous transplant. Alternatively, the transplanted cells are allogeneic.

Many techniques are known to those of ordinary skill in the art which can be used to help isolate, culture, induce differentiation, and to characterize the cells of the invention (Gorio et al., 2004, Neuroscience, 125:179-189; Yamashita et al., 2005, J. Cell Sci., 118:665-672; Conley et al., 2004, The International Journal of Biochemistry and Cell Biology, 36:555-567; Kindler, 2005, Journal of Leukocyte Biology, 78:836-844; Fuchs et al., 2004, Cell, 116:769-778; Campos, 2004, Journal of Neuroscience Research, 78:761-769; Dontu et al., 2005, Journal of Mammary Gland Biology and Neoplasia, 10:75-86).

### Examples

### Example 1

ASC Isolation, Culture Expansion and Characterization

My lab has extensive experience with the isolation, culture, and differentiation of human adipose-derived cells (Katz et al. 2005; Parker et al. 2007;Tholpady et al. 2005; Tholpady et al. 2003). We also have excellent experience in the use of flow cytometry to characterize ASCs (Katz et al. 2005).

### Example 2

Characterization of Human ASCs Formulated as 3-Dimensional Multicellular Aggregates (MAs)

An overview of methods and findings related to 3-D ASC culture includes the following:

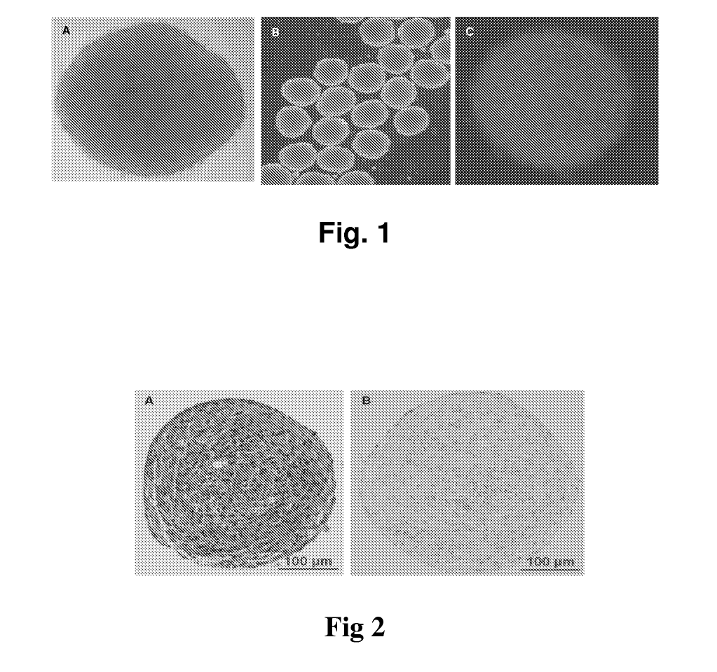

(1) ASCs can organize into 3-dimensional multicellular aggregates (MAs) in a controlled, reproducible fashion (FIGS. 1, 2); and

(2) they can be maintained for prolonged periods in culture and display robust survival capacity even when grown in serum-free conditions.

Methods: To reproducibly form cell aggregates, ASCs (500-50,000) were suspended in the appropriate medium to achieve desired concentrations. Small volumes (15-30 μl) of the concentrated cell suspensions were then pipetted onto culture plate covers in discrete droplets. The culture plate covers were then inverted, creating “hanging droplets”. After 24-72 hours in hanging drop culture, the MAs were then transferred to a range of media in either Ultra Low Attachment (ULA) wells/plates (Corning) for suspension culture, or into standard culture ware for adherent culture. In some experiments, ASC-MAs were labeled with Hoechst 33342 (Molecular Probes Cat#H1399) to reveal distribution of cell nuclei.

Results: Using a gravity-mediated technique, we demonstrate the successful and reproducible formation of 3-D ASC aggregates (MAs) using varied numbers of early passage ASCs (ranging from 500 to 50,000) isolated and cultured from individual donors (N>40). ASC MAs form, survive and grow in a variety of media types, including DMEM/F12 with 10% FBS (D-10), DMEM/F12 without serum or additives (D-0), serum-free ASC medium (AR8 and AR9), and low serum ASC medium (AR-1% HS and AR9-1% HS). FIG. 1a demonstrates the initial clustering and appearance of a typical ASC-MA soon after formation using a hanging drop technique. FIG. 1b demonstrates a photomicrograph of multiple well-defined, uniform sized MAs composed of fluorescently labeled (DiI) ASCs soon after their transfer to suspension culture.

(3) ASC MAs are composed of cells and self-generated extracellular matrix, enabling important cell-matrix interactions without the use of xenogeneic (or allogeneic) components (ex. bovine collagen); and (4) demonstrate growth and cellular proliferation in growth factor enriched medium.

Methods: In order to visualize cell migration and cell topography within MAS, MAs were formed with DiI labeled ASCs to enable easy visualization. BrdU pulsing studies were performed to characterize cell proliferation within MAs. MAs were fixed in formalin, incubated in 30% sucrose in PBS, and then flash frozen in cold isopentane. Cryostat sections of 8-micrometers were prepared for staining. Alternatively, MAs were embedded in paraffin and sectioned at 4 micrometers for analysis. Sections were stained with H&E, Oil Red O, Hoechst or Trichrome stain.

Results: Hoechst staining of whole MAs reveals extensive and relatively uniform cellularity on the outer surface of MAs (FIG. 1c). H&E staining reveals uniform cell (i.e. nuclei) distributed throughout the entire MA cross-section, embedded within hyaline-positive matrix (FIGS. 2 and 3). Trichrome staining reveals the presence of extensive collagen-based ECM (FIG. 2). MAs dramatically increase in size over time when placed in 3-D culture in growth factor supplemented serum-free medium (AR9). More specifically, under these conditions ASC-MAs grow in a unidirectional fashion, resulting in an elongated, oval-shape. BrdU immunostaining demonstrates an actively dividing non-fluorescent apical pole advancing away from a less proliferative brightly stained core (FIG. 3). This staining pattern suggests a quiescent, non-proliferating reservoir of cells within the MA milieu, as well as the ability to generate new cells when appropriately stimulated.

(5) ASC MAs demonstrate the capacity for adipogenic differentiation in vitro under a variety of conditions.

Methods: The adipogenic potential of ASC-MAs was evaluated in 3-D (suspension) culture, as well as after placement into adherent culture.

a. For suspension culture studies, human ASC MAs were grown in suspension in AR9 (serum free) medium for 2 weeks and then in adipogenic medium (AM) for 3 weeks. Control MAs were cultured in AR9 (growth) medium for a total of 5 weeks. After the 5-week culture period, MAs were frozen, cryosectioned, and stained with Oil Red O to detect lipid. Cohort studies were evaluated using RT-PCR measurement of adipose-specific genes.

b. For adherent culture studies, ASC MAs were formed and initially maintained in suspension, and then placed into adherent culture. When plated onto regular tissue culture plates, ASC MAs readily adhere to the culture surface, and, soon after adhesion generate progeny cells that will proliferate to monolayer confluence when maintained in supportive medium. In addition, adherent ‘source’ MAs can be repeatedly lifted from culture and transferred, or passaged, to a new culture vessel where they will readily attach and generate new progeny cells. To evaluate adipogenic differentiation of ASC MAs in Adherent Culture, MAs were cultured in suspension for 4 weeks. The MAs were then transferred to adherent culture. After 4 days in adherent culture, MA-derived ASCs were 2-D (monolayer) cultured for another 14 days in adipogenic medium (AM), or D-10 control medium. The cultures were then fixed and stained with Oil Red O.

c. To evaluate the adipogenic differentiation of ASCs derived from adherent, serially passaged MAs, the following studies were performed. ASC MAs were fabricated and 3-D cultured in AR9 medium for 12 days. They were then placed into adherent culture for 5-10 days in D-10 medium. Within 2-4 days of placement, adherent MAs were manually passaged by lifting them from the surface and placing them into new wells. After MAs were transferred (passaged) 10 times in this manner, cells that were ‘spawned’ from the P¹⁰ MAs were culture-expanded to confluent monolayers in D-10 medium for 6 weeks. These expanded P¹⁰ MA-derived cells were then cultured in adipogenic medium (AM) for 2 weeks, at which time they were fixed and stained with Oil Red-O.

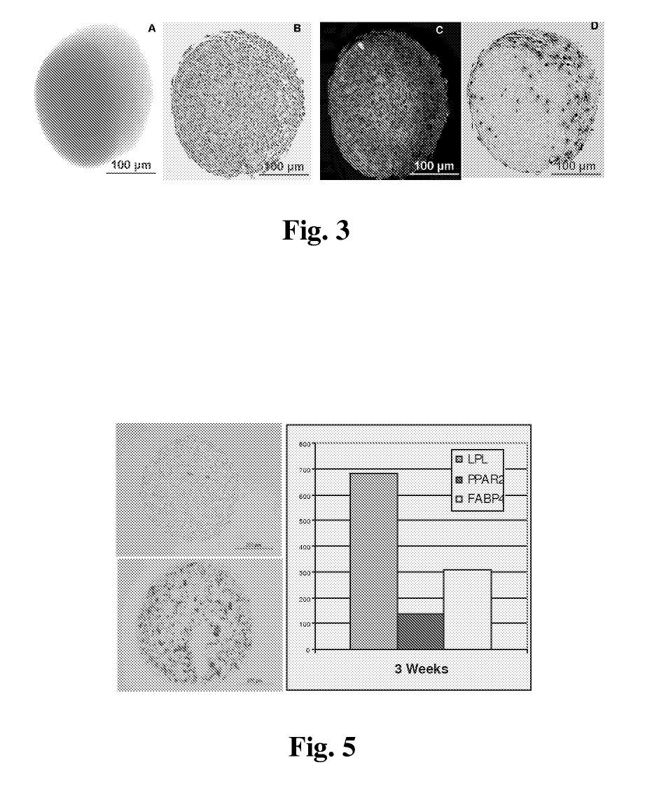

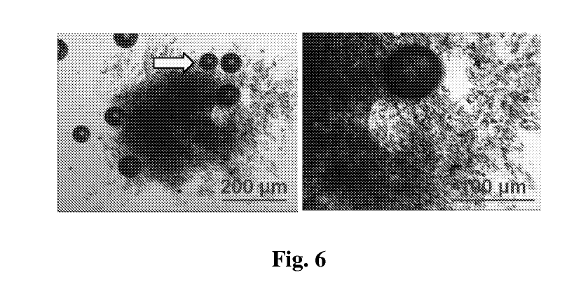

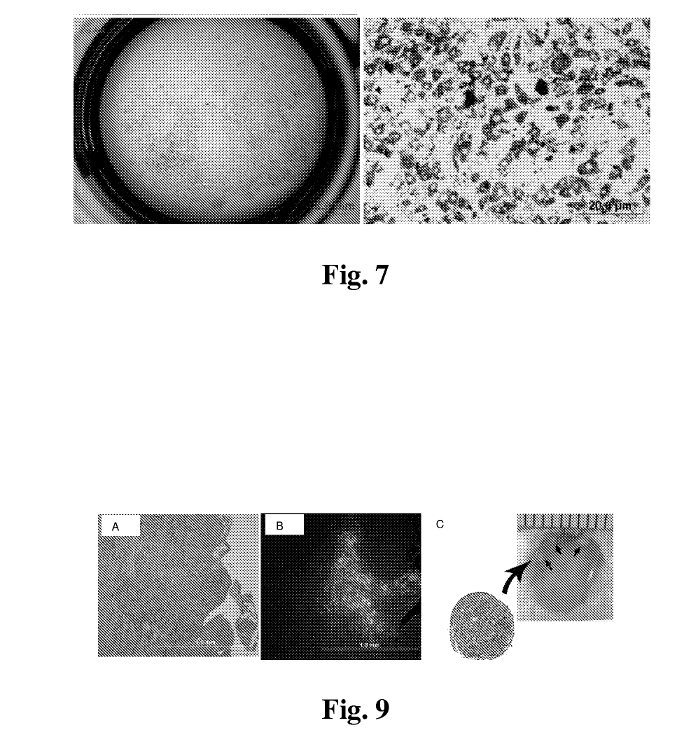

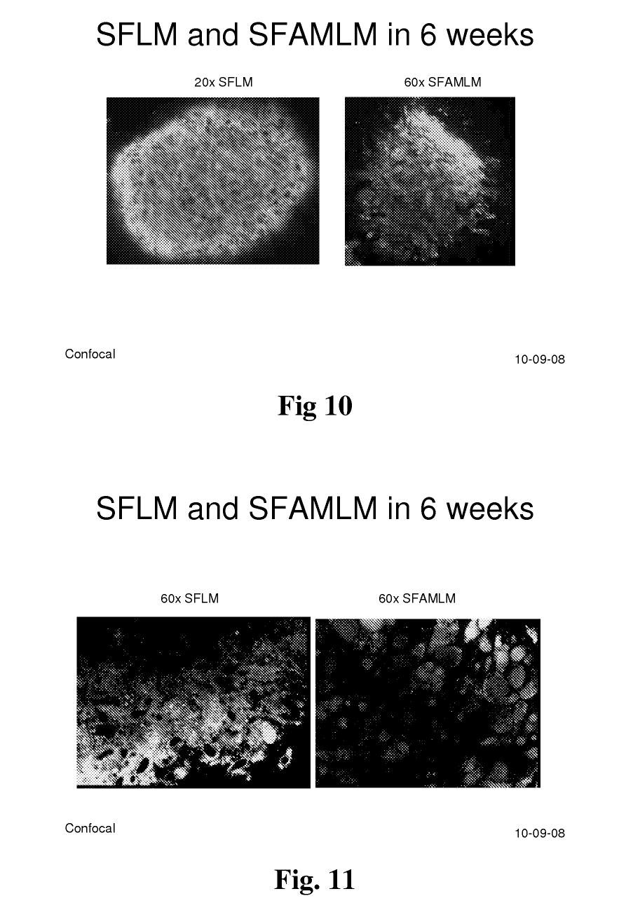

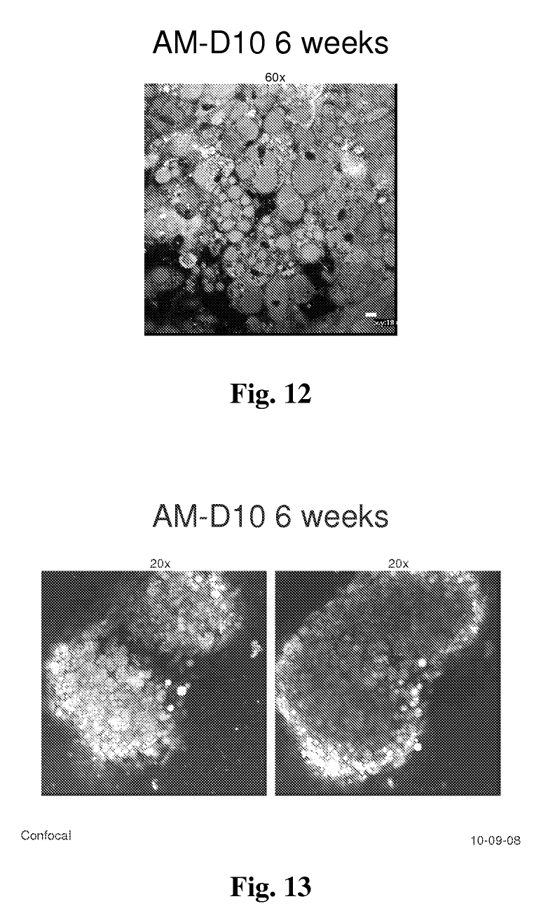

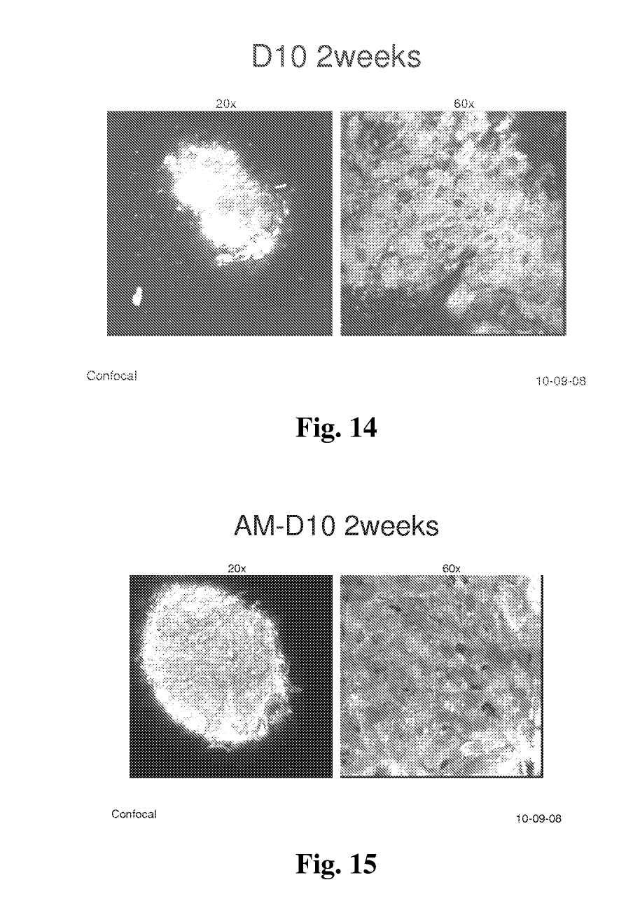

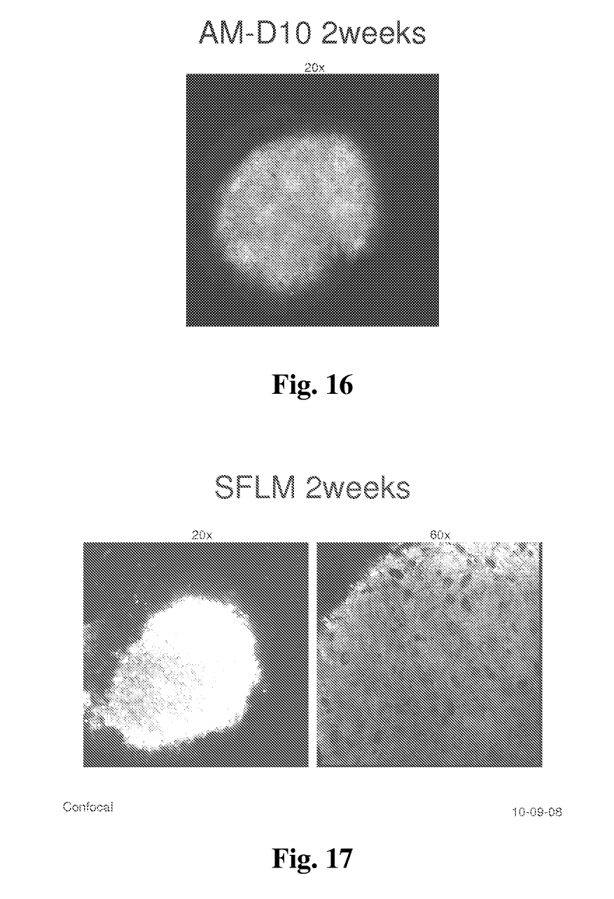

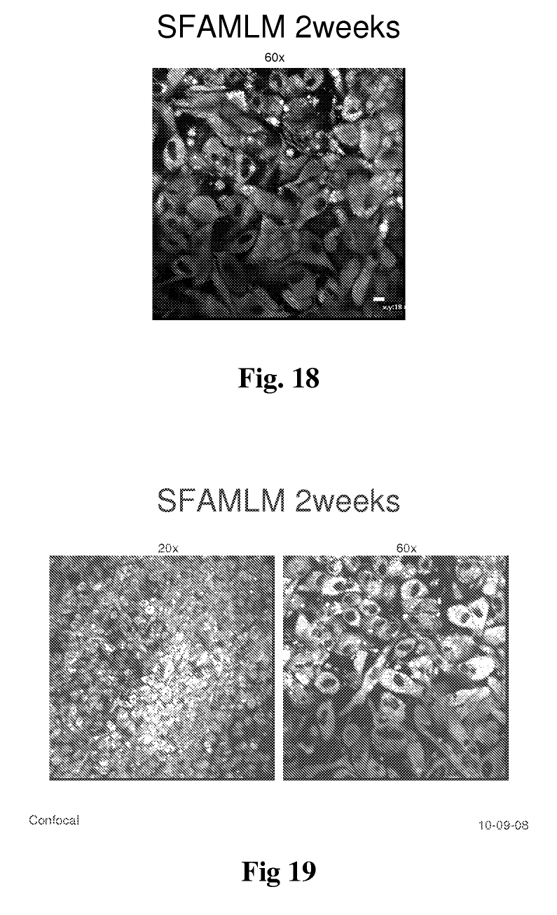

Results: MAs that are grown and induced in 3-D/suspension culture display staining characteristics consistent with adipogenic differentiation while control MAs grown under similar conditions but maintained in control medium do not (FIG. 5). PCR analysis demonstrates significant upregulation of adipogenic genes in MAs grown in adipogenic medium (FIG. 5). Similarly, monolayer ASCs derived from adherent cultured MAs readily undergo adipogenesis, even when the MAs have been cultivated in serum-free suspension culture prior to their adherence and induction (FIG. 6). ASCs derived from adherent, serially passaged MAs also maintain adipogenic plasticity despite prolonged culture and passaging (FIG. 7).

(6) ASC MAs produce bioactive factors that are relevant to tissue repair, angiogenesis, and adipogenesis; and, produce significantly higher levels of bioactive factors compared to ASCs grown in ‘traditional’ monolayer culture (FIG. 8). Methods: To determine whether ASC-MAs produce growth factors when maintained in suspension culture, freshly isolated ASCs were grown to confluence in adherent monolayer culture in DMEM-10% FBS (D10) medium. The cells were lifted into suspension and depleted of CD31+ and CD45+ cells using MACS columns (Miltenyi Biotech Cat #130-042-201) and anti-CD 31PE and anti-CD 45PE antibodies (BD Bioscience) and anti-PE microbeads (Miltenyi Biotech, #130-048-801). The passage 1 (P¹) CD31-/CD45-ASCs were then plated into monolayer culture at 2,000 cells/cm² in D10 medium. At near-confluence, the cells were again lifted into suspension and half were used to create 20,000 cell MAs, and the other half were kept in monolayer culture. After 24-48 hours in hanging drop, individual MAs were transferred to suspension culture in 6 well ULA (ultra low attachment) plates (Day 0) and maintained in one of 4 culture mediums: DMEM no serum (D-0), D-10, AR9 (chemically defined serum-free medium), or AR9-1% HS. For comparison, monolayer-cultured ASCs were (re)-plated at 2,000 cells/cm² into adherent monolayer culture using the same media conditions (Day 0). Cell culture supernatant was then collected and cell number quantified for each culture condition on post-plating day 3. The supernatant from each of 6 wells was combined and frozen for subsequent quantitative ELISA analysis of VEGF and HGF growth factor levels. Each sample (representing the combined supernatants from 6 separate but identical cultures) was analyzed in duplicate by Pierce Biotechnology's Searchlight™ service (www.piercenet.com/products), using appropriate standard curves for each analyte. Media-only controls served as ‘blanks’ and were subtracted from test values. Protein levels were normalized by dividing by the total number of cells for a given condition, resulting in picograms of growth factor/cell.

Results: Results demonstrate that ASC MAs make readily detectable levels of many growth factors, cytokines, and proteases (ex. VEGF, HGF, MCP2, MMP2). Compared to monolayer ASCs, MAs in 3-D suspension culture produce markedly elevated levels of most growth factors (in all 4 media conditions tested, data not shown. These results are consistent with the transcriptional upregulation of these same factors as detected in microarray analysis (Table 1), and support the hypothesis that 3-D MA culture confers enhanced biological potency compared to ASCs grown as adherent monolayers.

TABLE 1
ASC Upregulation of Select
Genes in 3-D Suspension Culture

| | Fold |
|---|---|
| | Change |
| Genes Corresponding to | |
| Secreted Proteins | |
| Measured by ELISA | |
| VEGF | ↑1.7x |
| HGF | ↑3.5x |
| E-Selectin | ↑2.4x |
| Insulin-like Growth Factor | ↑2x |
| Binding Protein 1 | |
| TGF beta 1 | ↑1.7x |
| TGF beta 2 | ↑1.4x |
| TIMP-1 | ↑1.7x |
| MMP-2 | ↑1.9x |
| Vascular Cell Adhesion | ↑5.5x |
| Molecule | |
| Adipose-Related Genes | |
| Fatty Acid Binding Protein | ↑4x |
| (FABP) | |
| Adiponectin | ↑2.5x |
| Peroxisome Proliferator | ↑1.9x |
| Activated Receptor gamma | |
| Perilipin | ↑2.5x |
| MMP-1 | ↑3.4x |

(7) ASC MAs differentially transcribe a substantial number of genes on a statistically significant level when compared to ASCs grown as monolayers. Methods: To evaluate the gene expression profile of ASC MAs relative to ASC monolayers, human ASCs were harvested and expanded as adherent monolayers in AR9-1% HS until sufficient cell numbers were obtained. The expanded cells were then divided into equal groups. One-half the cells were placed back into monolayer culture at 2,000 cells/cm2, while the remainder were formed into 25,000 cell MAs and transferred into suspension culture. Fresh medium was provided for all cultures on day 2-3. Between days 5 and 7, cells were harvested from both groups and RNA was isolated using a commercially available kit (PureLink Micro-ti-Midi Total RNA Purification System, Cat no. 12183-018). Monolayers and MAs from 3 different donors were analyzed for a total of 6 different specimens (3 monolayer specimens, 3 MA specimens). Each sample was tested in triplicate, each using a separate/individual Affymetrix human gene chip (HgU133 plus 2.0). Background intensity was derived from the intensity values of the lowest 2% of cells on the chip, establishing an overall baseline intensity that was subtracted from all cells before gene expression levels were calculated. Noise levels were derived from the standard deviation of the background intensity measurements. Affymetrix Gene Chip Operating Software (GCOS) was used to calculate signal intensity values as well as p-values and their associated presence calls for each sample. Expression data included the Affymetrix gene ID, signal intensity, detection p-value and detection call for each probe set on the chip. Two-group comparisons were carried out using dChip software.

Results: Comparison of MAs to monolayer cells revealed dramatic differences in gene expression, with MAs differentially expressing nearly 9,000 genes with statistical significance compared to monolayer cells. Many of these upregulated genes relate to adipogenic differentiation, and others reveal excellent correlation with protein expression levels measured by ELISA (Table 1). Statistical analysis of all samples analyzed (n=9 arrays for each group—monolayer cells and MAs) reveals that the gene expression profile of monolayer cells are over twice as variable as that of MAs, suggesting that the MA culture platform yields a more reproducible phenotype (‘product’) than monolayer methods.

(8) ASC MAs can be delivered in vivo directly from suspension culture as discrete “modular units” without disrupting cell homeostasis, cell-cell contacts, or cell-matrix interactions—as occurs with the trypsinization of adherent monolayer cells.

Methods: Human ASCs were isolated, culture expanded and formulated into MAs after membrane labeling with DiI fluorescent dye. MAs were then suspended in buffer and either injected into the left ventricular wall of mice, or pipetted into full thickness cutaneous wounds (separate experiments).

Results: ASCs survive injection as MAs into the cardiac environment, based on the visualization of DiI fluorescence on histological sections (FIG. 9). ASC MAs are grossly visible after delivery to open cutaneous wounds (FIG. 9).

The fundamental, overriding idea of this invention is that human adipose stromal/stem cells (ASCs) can predictably, reproducibly, and effectively generate new adipose tissue in vivo when fabricated and delivered as discrete 3-dimensional aggregates (MAs). Using methods developed in my lab, ASC MAs can be processed using completely defined conditions, making this platform extremely amenable to mechanistic studies and clinical translation.

In order for cells to generate adipose tissue in vivo, two fundamental conditions must be met: 1) the cells must survive after delivery in vivo, and 2) the cells must have adipogenic potential. This aim will specifically address the second of these fundamental conditions. Human ASCs are well known to have adipogenic capacity as adherent, monolayer cultures (Hauner et al. 1989; Hauner et al. 1987; van Harmelen et al. 2005; Wabitsch et al. 1995). Our studies have demonstrated the adipogenic capacity of ASCs as 3-D aggregates (MAs) maintained in suspension culture (section C.2.(5); FIG. 5)—including in defined, serum-free culture (i.e. induction) conditions. The use of serum (ex. 10% FBS)—such as that described in most, if not all of the published methods for ASC culture and differentiation—adds an additional source of capriciousness to a system that is already known to be variable due to the use of primary human cells (Schlesinger et al. 2006; Sen et al. 2001). Defined in vitro conditions and protocols are important for the reproducible and detailed analysis of adipose engineering, enabling targeted studies of mechanisms and the screening of adipogenic and anti-obesity therapeutics. And, from a translational perspective, defined serum-free methods may ultimately facilitate regulatory objectives that must be met for clinical use.

The more cells that survive and engraft after delivery in vivo, the greater the potential for therapeutic effect. I believe that ASCs formulated and delivered as MAs possess survival advantages over cells derived from, and delivered after ‘traditional’ adherent monolayer culture. Several findings from my lab support this hypothesis including: ASC MAs produce higher levels of cell survival and angiogenic factors (such as HGF and VEGF) that may help with initial cell survival and engraftment; and, they have well established cell-matrix interactions that may decrease anoikis-related cell loss after implantation. With more cells surviving implantation as MAs, it is reasonable to anticipate ‘better’ adipogenesis also. The 3-dimensional nature and dynamic, self-directed milieu of MAs is highly reminiscent of “cell clusters” described in the literature that initiate new adipose tissue formation in vivo (Crandall et al. 1997; Nishimura et al. 2007).

It is well accepted that the milieu of a given tissue dictates the behavior and form of its component cells. When a tissue/niche is in equilibrium, it is thought that the majority of stem cells are dormant/quiescent in the GO phase of the cell cycle (Kindler 2005). However, loss of, or damage to a tissue/niche provides a powerful stimulus to the stem cell reservoir to re-establish (i.e., repair; regenerate) equilibrium by renewal (expansion) and/or differentiation—all of which is thought to be governed by the (niche) micromilieu. For example, the type and structure of the extracellular matrix is known to exert important cues for adipogenic differentiation, and the ability of cells to remodel their physical environment is critical for the differentiation process to occur (Boudreau and Weaver 2006; Bouloumie et al. 2001; Bourlier et al. 2005; Mauney et al. 2005). ASCs represent the stem/progenitor cell fraction of adipose tissue and there is clear evidence that they readily differentiate along the adipogenic lineage under appropriate conditions. Matrix metalloproteinase 1 (MMP-1) has specifically been shown to play an integral role in this process in vivo (Boudreau and Weaver 2006). We have found that MMP-1 gene expression is upregulated over 3-fold in ASCs cultured as 3-D multicellular aggregates (Table 1). All things being equal, I believe that ASCs implanted into different recipient sites will demonstrate varying levels of survival and differentiation. More specifically, I believe that ASCs will survive and differentiate best when delivered into an adipose depot recipient site; and, delivery into a damaged or disrupted adipose site will accentuate this even further, due to signals released by the host tissue/environment to re-establish homeostatic equilibrium.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Aust, L, Devlin, B, Foster, S J, Halvorsen, Y D, Hicok, K, du Laney, T, Sen, A, Willingmyre, G D and Gimble, J M (2004). “Yield of human adipose-derived adult stem cells from liposuction aspirates.” Cytotherapy 6(1): 7-14.

2. Boudreau, N and Weaver, V (2006). “Forcing the third dimension.” Cell 125(3): 429-31.

3. Bouloumie, A, Sengenes, C, Portolan, G, Galitzky, J and Lafontan, M (2001). “Adipocyte produces matrix metalloproteinases 2 and 9: involvement in adipose differentiation.” Diabetes 50(9): 2080-6.

4. Bourlier, V, Zakaroff-Girard, A, De Barros, S, Pizzacalla, C, de Saint Front, V D, Lafontan, M, Bouloumie, A and Galitzky, J (2005). “Protease inhibitor treatments reveal specific involvement of matrix metalloproteinase-9 in human adipocyte differentiation.” J Pharmacol Exp Ther 312(3): 1272-9.

5. Cao, Y (2007). “Angiogenesis modulates adipogenesis and obesity.” J Clin Invest 117(9): 2362-8.

6. Cho, S W, Kim, I, Kim, S H, Rhie, J W, Choi, C Y and Kim, B S (2006). “Enhancement of adipose tissue formation by implantation of adipogenic-differentiated preadipocytes.” Biochem Biophys Res Commun 345(2): 588-94.

7. Choi, Y S, Park, S N and Suh, H (2005). “Adipose tissue engineering using mesenchymal stem cells attached to injectable PLGA spheres.” Biomaterials 26(29): 5855-63.

8. Crandall, D L, Hausman, G J and Kral, J G (1997). “A review of the microcirculation of adipose tissue: anatomic, metabolic, and angiogenic perspectives.” Microcirculation 4(2): 211-32.

9. Eremia, S and Newman, N (2000). “Long-term follow-up after autologous fat grafting: analysis of results from 116 patients followed at least 12 months after receiving the last of a minimum of two treatments.” Dermatol Surg 26(12): 1150-8.

10. Fuchs, E, Tumbar, T and Guasch, G (2004). “Socializing with the neighbors: stem cells and their niche.” Cell 116(6): 769-78.

11. Fukumura, D, Ushiyama, A, Duda, D G, Xu, L, Tam, J, Krishna, V, Chatterjee, K, Garkavtsev, I and Jain, R K (2003). “Paracrine regulation of angiogenesis and adipocyte differentiation during in vivo adipogenesis.” Circ Res 93(9): e88-97.

12. Gimble, J M, Katz, A J and Bunnell, B A (2007). “Adipose-derived stem cells for regenerative medicine.” Circulation Research 100(9): 1249-60.

13. Gronthos, S, Franklin, D M, Leddy, H A, Robey, P G, Storms, R W and Gimble, J M (2001). “Surface protein characterization of human adipose tissue-derived stromal cells.” J Cell Physiol 189(1): 54-63.

14. Halbleib, M, Skurk, T, de Luca, C, von Heimburg, D and Hauner, H (2003). “Tissue engineering of white adipose tissue using hyaluronic acid-based scaffolds. I: in vitro differentiation of human adipocyte precursor cells on scaffolds.” Biomaterials 24(18): 3125-32.

15. Hauner, H, Entenmann, G, Wabitsch, M, Gaillard, D, Ailhaud, G, Negrel, R and Pfeiffer, E F (1989). “Promoting effect of glucocorticoids on the differentiation of human adipocyte precursor cells cultured in a chemically defined medium.” J Clin Invest 84(5): 1663-70.

16. Hauner, H, Schmid, P and Pfeiffer, E F (1987). “Glucocorticoids and insulin promote the differentiation of human adipocyte precursor cells into fat cells.” J Clin Endocrinol Metab 64(4): 832-5.

17. Hemmrich, K and von Heimburg, D (2006). “Biomaterials for adipose tissue engineering.” Expert Rev Med Devices 3(5): 635-45.

18. Hong, L, Peptan, I A, Colpan, A and Daw, J L (2006). “Adipose tissue engineering by human adipose-derived stromal cells.” Cells Tissues Organs 183(3): 133-40.

19. Kanchwala, S K and Bucky, L P (2003). “Facial fat grafting: the search for predictable results.” Facial Plast Surg 19(1): 137-46.

20. Katz, A J, Tholpady, A, Tholpady, S S, Shang, H and Ogle, R C (2005). “Cell surface and transcriptional characterization of human adipose-derived adherent stromal (hADAS) cells.” Stem Cells 23(3): 412-23.

21. Katz, A J, Yang, Z, Shang, H, Chamberlain, A T, Berr, S S, Roy, R J, Khurgel, M, Epstein, F H and French, B A (2006). “Serial MRI Assessment of Hman Adipose-derived Stem Cells (hASCs) in a Murine Model of Reperfused Myocardial Infarction.” Adipocytes 2(1).

22. Kaufman, M R, Bradley, J P, Dickinson, B, Heller, J B, Wasson, K, O'Hara, C, Huang, C, Gabbay, J, Ghadjar, K and Miller, T A (2007). “Autologous fat transfer national consensus survey: trends in techniques for harvest, preparation, and application, and perception of short- and long-term results.” Plast Reconstr Surg 119(1): 323-31.

23. Khurgel, M, Shang, H, Parker, A M and Katz, A J (2006). 3-Dimensional Suspension Culture of Human Adipose Stromal Cells: Self-Organizing, Self-Sustained Dynamic Cell Niches. 4th Annual Meeting of the International Federation of Adipose Therapeutics and Science (IFATS). Baton Rouge, La.

24. Kimura, Y, Ozeki, M, Inamoto, T and Tabata, Y (2003). “Adipose tissue engineering based on human preadipocytes combined with gelatin microspheres containing basic fibroblast growth factor.” Biomaterials 24(14): 2513-21.

25. Kindler, V (2005). “Postnatal stem cell survival: does the niche, a rare harbor where to resist the ebb tide of differentiation, also provide lineage-specific instructions?” Journal of Leukocyte Biology 78(4): 836-44.

26. Kumanyika, S K (2007). “The obesity epidemic: looking in the mirror.” Am J Epidemiol 166(3): 243-5.

27. Lee, S T, Chu, K, Kim, E H, Jung, K H, Lee, K B, Sinn, D I, Kim, S U, Kim, M and Roh, J K (2006). “Quantification of human neural stem cell engraftments in rat brains using ERV-3 real-time PCR.” J Neurosci Methods 157(2): 225-9.

28. Lijnen, H R (2007). “Angiogenesis and obesity.” Cardiovasc Res.

29. Lower, R, Lower, J and Kurth, R (1996). “The viruses in all of us: characteristics and biological significance of human endogenous retrovirus sequences.” Proc Natl Acad Sci USA 93(11): 5177-84.

30. Mauney, J R, Volloch, V and Kaplan, D L (2005). “Matrix-mediated retention of adipogenic differentiation potential by human adult bone marrow-derived mesenchymal stem cells during ex vivo expansion.” Biomaterials 26(31): 6167-75.

31. Missana, M C, Laurent, I, Barreau, L and Balleyguier, C (2007). “Autologous fat transfer in reconstructive breast surgery: indications, technique and results.” Eur J Surg Oncol 33(6): 685-90.

32. Mitchell, J B, McIntosh, K, Zvonic, S, Garrett, S, Floyd, Z E, Kloster, A, Di Halvorsen, Y, Storms, R W, Goh, B, Kilroy, G, Wu, X and Gimble, J M (2006). “Immunophenotype of human adipose-derived cells: temporal changes in stromal-associated and stem cell-associated markers.” Stem Cells 24(2): 376-85.

33. Nishimura, S, Manabe, I, Nagasaki, M, Hosoya, Y, Yamashita, H, Fujita, H, Ohsugi, M, Tobe, K, Kadowaki, T, Nagai, R and Sugiura, S (2007). “Adipogenesis in obesity requires close interplay between differentiating adipocytes, stromal cells, and blood vessels.” Diabetes 56(6): 1517-26.

34. Parker, A M and Katz, A J (2006). “Adipose-derived stem cells for the regeneration of damaged tissues.” Expert Opinion on Biological Therapy 6(6): 567-78.

35. Parker, A M, Shang, H, Khurgel, M and Katz, A J (2007). “Low serum and serum free culture of multipotential human adipose stem cells.” Cytotherapy 9(7): 637-646.

36. Prichard, H L, Reichert, W M and Klitzman, B (2007). “Adult adipose-derived stem cell attachment to biomaterials.” Biomaterials 28(6): 936-46.

37. Rehman, J, Traktuev, D, Li, J, Merfeld-Clauss, S, Temm-Grove, C J, Bovenkerk, J E, Pell, C L, Johnstone, B H, Considine, R V and March, K L (2004). “Secretion of angiogenic and antiapoptotic factors by human adipose stromal cells.” Circulation 109(10): 1292-8.

38. Schaffler, A and Buehler, C (2007). “Concise review: adipose tissue-derived stromal cells—basic and clinical implications for novel cell-based therapies.” Stem Cells 25(4): 818-27.

39. Schlesinger, J B, van Harmelen, V, Alberti-Huber, C E and Hauner, H (2006). “Albumin inhibits adipogenesis and stimulates cytokine release from human adipocytes.” Am J Physiol Cell Physiol 291(1): C27-33.

40. Sen, A, Lea-Currie, Y R, Sujkowska, D, Franklin, D M, Wilkison, W O, Halvorsen, Y D and Gimble, J M (2001). “Adipogenic potential of human adipose derived stromal cells from multiple donors is heterogeneous.” J Cell Biochem 81(2): 312-9.

41. Shenaq, S M and Yuksel, E (2002). “New research in breast reconstruction: adipose tissue engineering.” Clin Plast Surg 29(1): 111-25, vi.

42. Tholpady, S S, Aojanepong, C, Llull, R, Jeong, J-H, Mason, A C, Futrell, J W, Ogle, R C and Katz, A J (2005). “The cellular plasticity of human adipocytes.” Annals of Plastic Surgery 54(6): 651-6.

43. Tholpady, S S, Katz, A J and Ogle, R C (2003). “Mesenchymal stem cells from rat visceral fat exhibit multipotential differentiation in vitro.” Anatomical Record Part A, Discoveries in Molecular, Cellular, & Evolutionary Biology 272(1): 398-402.

44. van Harmelen, V, Skurk, T and Hauner, H (2005). “Primary culture and differentiation of human adipocyte precursor cells.” Methods Mol Med 107: 125-35.

45. Varma, M J, Breuls, R G, Schouten, T E, Jurgens, W J, Bontkes, H J, Schuurhuis, G J, van Ham, S M and van Milligen, F J (2007). “Phenotypical and functional characterization of freshly isolated adipose tissue-derived stem cells.” Stem Cells Dev 16(1): 91-104.

46. Wabitsch, M, Hauner, H, Heinze, E and Teller, W M (1995). “The role of growth hormone/insulin-like growth factors in adipocyte differentiation.” Metabolism 44(10 Suppl 4): 45-9.

47. Wang, Y and Beydoun, M A (2007). “The obesity epidemic in the United States—gender, age, socioeconomic, racial/ethnic, and geographic characteristics: a systematic review and meta-regression analysis.” Epidemiol Rev 29: 6-28.

48. Wilmore, J H (2007). “The obesity epidemic.” Curr Sports Med Rep 6(6): 345-6.

49. Yuan, C C, Miley, W and Waters, D (2001). “A quantification of human cells using an ERV-3 real time PCR assay.” J Virol Methods 91(2): 109-17.

50. Zuk, P A, Zhu, M, Mizuno, H, Huang, J, Futrell, J W, Katz, A J, Benhaim, P, Lorenz, H P and Hedrick, M H (2001). “Multilineage cells from human adipose tissue: implications for cell-based therapies.” Tissue Engineering 7(2): 211-28.

1. A method of making a 3-dimensional modular adipogenic construct, the modular adipogenic construct comprising cellular components with adipogenic potential, said method having the steps of:
a. harvesting adipose tissue from a mammalian subject;
b. isolating adipose tissue-derived stromal cells from the harvested adipose tissue;
c. culturing the isolated cells in 3-dimensional multicellular aggregates in a controlled, reproducible fashion such that the 3-dimensional multicellular aggregate self-generates an extracellular matrix that is serum-free, free of exogenous materials, zenogeneic-free, and free of synthetic components.

2. The method of claim 1 wherein the cellular components with adipogenic potential are adipose stem cells.

3. The method of claim 1 wherein the cellular components with adipogenic potential are selected from the group consisting of adipose stem cells, stromal cells, and progenitor cells.

4. A method of using 3-dimensional modular adipogenic constructs for therapeutic purposes, including tissue repair, tissue regeneration, tissue replacement, or tissue augmentation, said method having the steps of:
a. generating 3-dimensional modular adipogenic constructs;
b. implanting said modular adipogenic constructs in the tissues of a patient.

5. The method of claim 4 wherein the modular adipogenic construct is combined with growth factors, antibiotics, or cytokines.

6. The method of claim 4 wherein the adipogenic construct is combined with a gel, scaffold, or exogenous factor.
